(19) United States
(12) Patent Application Publication
(10) Pub. No.: US 2007/0237809 A1
(43) Pub. Date: Oct. 11, 2007

(54) Multi-functional bioactive wound dressing

(76) Inventors: Matthew D. Phaneuf, Ashland, MA (US); Susan L. Hannel, North Kingston, RI (US); Martin J. Bide, S. Kingstown, RI (US); Frank W. LoGerfo, Cambridge, MA (US)

Correspondence Address: David Prashker;DAVID PRASHKER, P.C., P.O. Box 5387, Magnolia, MA, 01930, US

(21) Appl. No.: 11/211,936

(22) Filed: Aug. 25, 2005

Related U.S. Application Data
(60) Provisional application No. 60/605,627, filed on Aug. 30, 2004.

(51) Int. Cl. A61L 15/00; B32B 27/04

(52) U.S. Cl. 424/445; 442/123

(57) ABSTRACT

A novel, lightweight bioactive wound dressing is provided which offers infection-resistance as well as localized wound healing properties. The wound dressing is comprised of preformed fibrous matter, which is comprised of either a composition which intrinsically provides multiple chemical groups which are functional for subsequent chemical reaction; or is a composition which has been chemically modified at its exterior surface to present a range of chemical groups which are functional for subsequent chemical reaction. These multiple functional chemical groups serve as a plurality of anchorage sites and backbone for the subsequent immobilization of an independently bound biologically active protein. Preferably, a broad-spectrum antibiotic such as the fluoroquinolone, ciprofloxacin, is incorporated into the preformed fibrous matter, using a newly created Pad/Autoclaving technique. Following antibiotic incorporation, the exterior surface of the fibrous matter has a biologically-active protein—such as thrombin, a pivotal enzyme in the blood coagulation cascade—covalently attached and permanently bound to the exterior surface as an aid for enhanced clotting propensity and to help establish hemostasis in-vivo.

### PRIORITY CLAIM

The present invention was first filed as U.S. Provisional Patent Application Ser. No. 60/605,627 on Aug. 30, 2004. The priority and legal benefit of this first filing is expressly claimed

### FIELD OF THE INVENTION

The present invention is concerned generally with improvements in wound dressings; and is specifically directed to a single article wound dressing which would provide a synergistic combination of specific physical properties (such as adjustable compression, individual application, durability, compact, and ease of application) as well as provide particular biological properties such as infection-resistance and enhanced wound healing in-vivo. The wound dressing comprising the instant invention offers a marked resistance to infection as well as a localized biologic activity in-situ to stimulate hemostasis—attributes which are instrumental in reducing both the morbidity and mortality of a person having peripheral injuries.

### BACKGROUND OF THE INVENTION

Trauma—whether caused by a motor vehicle accident, pedestrian accident, accidental firearm discharge, recreational accident, criminal act, terrorist act or battlefield conditions—results in significant morbidity and mortality. In 2002, over 400,000 trauma cases were reported in the United States, with some 148,000 Americans dying each year. Of these mortalities, 40% have been attributed to uncontrolled bleeding at the trauma site; and overall, these traumas have resulted in a total cost of $260 billion to the healthcare system, accounting for 12% of all medical spending.

Any penetration of the human body carries with it the risk of potential infection by microbes. This risk pertains particularly to traumatic wounds incurred by accident or negligence; to wound treatment procedures which utilize different kinds of materials for closure of the wound; to the different kinds of dressings available for skin penetrations and/or body wounds; and to a diverse range of health care products which are introduced to the body for therapeutic and/or prophylactic purposes.

The rational use of antimicrobial agents against infection, particularly for wound treatment, has been advocated generally, and such use has been previously reviewed in detail within the medical literature [see for example., Rodgers, K. G., Emer. Med. Clin. N. Am. 10: 753 (1992)]. Similarly, the major concerns regarding the ever-growing incidence of infections resulting from the use of biomedical textiles, fabrics and fiber containing articles and devices—despite recent advances in sterile procedures used in the clinical/surgical setting—have been recognized and considered to be of primary importance [see for example, the FDA/EPA/CDC/AAMI joint conference in Proceedings, Infection Control Symposium: Influence Of Medical Device Design, U.S. Dept. of Health and Human Services, Bethesda, Md., January 1995]. Moreover, the use of antibiotics and of mechanisms for delivering antimicrobial agents generally, particularly via slow-release delivery systems over time, to prevent or reduce the severity of infection for implanted biodegradable materials has become prominent [see for example, Sasmor et al., J Vasc. Sur. 14: 521 (1993)]. All of these developments and considerations lead to the same conclusion: Infection, with or without the use of antibiotics, must be prevented or be controlled for all textile fiber containing materials regardless of clinical need or medical purpose.

### Recent Efforts to Combat Infections by Biomaterials

Strategies

Numerous strategies have been proposed and attempted in order to create an infection-preventing surface for biomaterials. Much of this effort has been directed at surgically implantable textiles and in-vivo engraftable articles. However, these efforts to reduce and to combat surgical infections in-vivo are merely a representative portion of the greater problem as a whole directed towards biomaterials comprised of fibrous matter which are able to prevent and interdict infections generally—i.e., without regard to whether or not the potential infection is airborne, topical, percutaneous or subcutaneous, humoral, and organ or tissue specific.

For example, a variety of different chelating agents have been evaluated for use as a release system for antibiotics from a biomaterial surface. One favored approach has been the ionic binding of antibiotics by surfactants. Cationic surfactants (such as tridodecylmethyl ammonium chloride and benzalkonium chloride) were sorbed at the anionic surface potential of a polymeric material, thereby achieving a weak adhesion of anionic antibiotics to the surface of the polymer [see for example: Harvey et al., Ann. Surg. 194: 642 (1981); Harvey et al., Surgery 92: 504 (1982); Harvey et al., Am. J. Surg. 147: 205 (1984); Shue et al., J. Vasc. Surg. 8: 600 (1988); and Webb et al., J. Vasc. Surg. 4: 16 (1956)]. The surfactant immobilized antibiotic subsequently was released into mobile form upon contact with blood.

Silver was also examined as a release system for various antibiotics from textile surfaces. Silver was applied either as a chelating agent [see for example: Modak et al., Surg. Gynecol. Obstet. 164: 143 (1987); Benvenisty et al., J. Surg. Res. 44: 1 (1988); and White et al., J. Vasc. Surg. 1: 372 (1984)], or alone in metallic form, for its antimicrobial properties.

Another favored approach has employed various binding agents in order to create localized concentrations of an antibiotic on the article's surface. These binding agents, typically a protein or a synthetic-based substance, were embedded within the biomaterial matrix, thereby either “trapping” or ionically binding with the antibiotic of choice. In this manner, the basement membrane protein collagen has often served as a binding agent and as a release system for rifampin, a system demonstrated to have antimicrobial efficacy in a bacteremic challenge dog model [Krajicek et al., J. Cardiovasc. Surg. 10: 453 (1969)] as well as in early European clinical trials [Goeau-Brissonniere, O., J. Mal. Vasc. 21: 146 (1996); Strachan et al., Eur. J. Vasc. Surg. 5: 627 (1991)].

Similarly, fibrin, present either as a glue or as a factor in pre-clotted blood, has been utilized as a binding agent for the immobilization of various antibiotics, including gentamycin, rifampin and tobramycin [see for example, Haverich et al., J. Vasc. Surg. 14: 187 (1992); McDougal et al., J. Vasc. Surg. 4: 5 (1986); Powell et al., Surgery 94: 765 (1983); Greco et al., J Biomed. Mater. Res. 25: 39 (1991)].

Furthermore, Levofloxacin (itself a quinolone, a synthetic analog of nalidixic acid) has been incorporated in an albumin matrix and gelatin has been used as the release system for the antibiotics rifampin and vancomycin, with animal studies also showing efficacy in acute bacteremic challenges [see for example, Muhl et al., Ann. Vasc. Surg. 10: 244 (1996); Sandelic et al., Cardiovasc. Surg. 4: 389 (1990)].

In addition to the foregoing, a variety of synthetic binding agents have also been evaluated for antibiotic release as a replacement for the naturally occurring protein binders. Some synthetic binders were incorporated directly into the biomaterial matrix (in a similar fashion to the protein binders) which permitted a sustained release of a selected antibiotic over time [see Shenk et al., J Surg. Res. 47: 487 (1989)]. Recent techniques also have utilized these types of synthetic binder materials as a scaffolding to bind antibiotics covalently to the biomaterial surface [see Suzuki et al., ASAIO J. 43: M854 (1997)]. Release of the antimicrobial agent was controlled by bacterial adhesion to the surface, which resulted in antibiotic cleavage and release. This mechanism of activity promotes “bacterial suicide” while maintaining antibiotic concentration, which is not needed to prevent infection, localized on the surface.

Other techniques have involved which incorporate the antibiotic either into the process of synthesizing the polymer [see Golomb et al., J. Biomed. Mater. Res. 25: 937 (1991); Whalen et al., ASAIO J. 43: M842 (1997); or embed the antibiotic directly into the interstices of the material [Okahara et al., Eur. J. Vasc. Endovasc. Surg. 9: 408 (1995)].

Recognized Drawbacks and Complications

It will be recognized and appreciated also that there are several serious drawbacks and undesirable complications in effect for each of these individual antibiotic immobilization strategies. For the approach using chelation agents, 50% of the antibiotic has been shown to elute from the graft surface within 48 hours, with less than 5% antibiotic remaining after three weeks [see Greco et al., Arch. Surg. 120: 71 (1985)]. While this degree of antibiotic coverage is adequate for small localized contaminations, it is clear that large infectious inoculums are not addressed. In contrast, with the approach using binding agents, antibiotic release often is quite varied and will depend on the rate of binder degradation or binder release from a surface which is under high shear stress from blood flow. Comparably, both types of surface modifications rely on exogenous matter which may affect the overall properties of the textile surface, either by releasing toxic moieties or by promoting thrombogenesis. Thus, these potential complications have accentuated the need to create an infection-preventing textile fabric surface which is devoid of exogenous matter such as binding agents.

### Use of Antibiotics as Dyes

Noticeably, all of the above identified reported investigations avoid the examination of any direct material/antibiotic interaction. However, some attempts have been recently made to use direct interactions, particularly dye-fiber interactions, as a model; and these systems can provide infection resistance without the use of exogenous binders. It is essential also to note that, unlike proteins which are still active after being covalently bound, subsequent antibiotic release and mobility of the antibiotic itself is essential if antimicrobial activity is to occur in-situ. Moreover, dyes have substantivity; and dyes will “exhaust” from a bath preferentially into a fiber, when drawn by physical forces of attraction.

Initial efforts in this regard examined the use of commercially available dyes as anchors for antibiotic molecules, and even determined the antibiotic activity of some dyes [see for example: U.S. Pat. No. 5,281,662; and Bide et al., Textile Chemist and Colorist 25: 15-19 (1993). This approach, however, was found to be unrewarding.

Subsequently, in contrast to these initial investigations, the direct use of antibiotics was then examined [see for example: Phaneuf et al., J. Biomed. Mat. Res. 27: 233-237 (1993); Ozaki et al., J. Surg. Res. 55: 543-547 (1993); Phaneuf et al., in Antimicrobial/Anti-Infective Materials (Sawan, S. P. and G. Manivannan, editors), Chap. 10, pp. 239-259 (2000); and the references cited within each of these printed publications].

Fluoroquinolone antibiotics were found to be particularly suitable in such applications. Fluoroquinolones, as a discrete family of chemical compounds, are all stable to dry heat and to hot aqueous media; they all have an appropriate molecular size, and (in the absence of any reliable method for predicting physical interactions) they all share a somewhat dye-like structure. Two of the most common commercially manufactured quinolones which are currently available are Ciprofloxacin (hereinafter “Cipro”) and Ofloxacin (hereinafter “Oflox”).

Nevertheless, despite all these developments to date, there remains a recognized and continuing need for further improvements in the making of infection preventing wound dressings effective against the infiltration of microbes, and which would serve as biomedical constructs formed of fibrous materials having demonstrable antimicrobial properties. All such improvements in the making and/or preparation of wound dressing articles which can treat and/or prevent microbial infections in-vivo and preclude microbial growth in-situ would be seen as a major advantage and outstanding benefit in this field.

### SUMMARY OF THE INVENTION

The present invention has multiple aspects.

A first aspect provides a fabric article useful as a wound dressing, said fabric article comprising:

preformed fibrous matter comprised of at least one chemical composition which provides multiple chemical groups functional for subsequent chemical reaction, said fibrous matter being of determinable dimensions and configuration, having at least one exterior surface which presents a plurality of functional chemical groups for chemical reaction, and being formed of at least one type of material able to take up aqueous fluids;

at least one water-miscible antibiotic composition of fixed concentration which is incorporated into said fibrous matter in the dry state, but which becomes detached from said fibrous matter and is released into the ambient environment as a freely mobile antibiotic composition after said fibrous matter takes up an aqueous fluid, said antibiotic composition having recognized characteristic antimicrobial properties in the immobilized and freely mobile forms, being a heat stable substance, and having a relative molecular mass in the 300-1500 range; and

a prechosen biologically active protein covalently bound to said fibrous matter, said protein having recognized biologically active properties for aiding the wound healing process while bound to said fibrous matter.

A second aspect of the invention provides a fabric article useful as a wound dressing, said fabric article comprising:

preformed fibrous matter of predetermined dimensions and configuration, said fibrous matter having at least one exterior surface and being formed of at least one type of material able to take up aqueous fluids;

a coupling agent attached to at least the exterior surface portion of said preformed fibrous matter;

at least one water-miscible antibiotic composition of fixed concentration which is incorporated into said fibrous matter in the dry state, but which becomes detached from said fibrous matter and is released into the ambient environment as a freely mobile antibiotic composition after said fibrous matter takes up an aqueous fluid, said antibiotic composition having recognized characteristic antimicrobial properties in the immobilized and freely mobile forms, being a heat stable substance, and having a relative molecular mass in the 300-1500 range;

at least one bifunctional cross-linking molecule joined to said coupling agent at said exterior surface of said fibrous matter; and

a prechosen biologically active protein covalently bound by said bifunctional cross-linking molecule to said fibrous matter, said protein having recognized biologically active properties for aiding the wound healing process while bound to said fibrous matter.

### BRIEF DESCRIPTION OF THE FIGURES

The present invention may be better understood and more readily appreciated when taken in conjunction with the accompanying Drawing, in which:

FIG. 1 is a chart showing the exposure of D-PU material to ethylenediamine in order to create both carboxylic acid and amine groups within the polymer structure as evidenced by methylene blue and CI Acid Red;

FIG. 2 is a graph showing that bD-PU-AB segments had significant antimicrobial activity as evaluated by the zone inhibition assay;

FIG. 3 is a graph showing that bD-PU-AB segments with covalently immobilized ¹²⁵I-thrombin had substantial antimicrobial activity when compared to other, differently prepared test segments;

FIG. 4 is a graph showing that bD-PU-AB segments with covalently immobilized ¹²⁵I-thrombin had a markedly increased surface thrombin activity when compared to other, differently prepared test segments;

FIG. 5 is an overhead view of the surface thrombus deposition that was macroscopically apparent on bd-PU-AB-Thrombin segments after exposure to whole blood;

FIG. 6 is a set of scanning electron microscopic images showing the time sequence of thrombus formation on the surface of bd-PU-AB-Thrombin segments; and

FIGS. 7A, 7B, and 7C are individual views of a novel compression wound dressing article.

### DETAILED DESCRIPTION OF THE PRESENT INVENTION

The present invention is a novel, lightweight bioactive compression wound dressing that provides infection-resistance via a releasable antibiotic incorporated into the fibrous structure of a preformed fibrous matter, as well as provides localized wound healing properties which are obtained via the presence of a biologically-active protein. One preferred embodiment utilizes polyester with polyurethane inlayed into the fibrous structure as the preformed fibrous matter of the wound dressing; and this blend of synthetics provides a range of highly desired physical properties (e.g., elasticity, durability) for the dressing as a whole.

In this preferred polyester/polyurethane embodiment, the preformed fibrous matter has been chemically modified in the following ways: initially, by using a diamine coupling agent to join multiple reactive chemical groups to the surface of the material, and to generate a modified surface which then provides a plurality of anchor sites for subsequent protein attachment. Next, by incorporating a broad-spectrum antibiotic, such as the fluoroquinolone, ciprofloxacin, as a releasable entity onto the exterior surface of said modified fibrous material using a newly developed pad/autoclave process. Then, by using one or more bifunctional linking agents which are able to react with the functional chemical groups already present on the modified surface of the fibrous matter, and are used for the permanent immobilization and covalent attachment of a protein for enhanced wound healing. In this manner, a biologically-active protein (such as thrombin) becomes covalently bound to the preformed fibrous matter together with an incorporated, but releasable, antibiotic. Overall therefore, this procedure as a whole yields a fibrous matter wound dressing having a temporarily incorporated antibiotic, which will be released into mobile form upon exposure to an aqueous environment and will provide localized antimicrobial properties in-situ; and, concurrently, having an enhanced clotting propensity in-situ via the covalently joined biologically active protein, which will help to establish hemostasis in-vivo.

### Wording, Terminology and Titles

Although many of the words, terms and titles employed herein are commonly used and conventionally understood within its traditional medical usage and scientific context, a summary description and definition is presented below for some phrases and wording as well as for particular names, designations, epithets or appellations. These descriptions and definitions are provided as an aid and guide to recognizing and appreciating the true variety and range of applications intended for inclusion within the scope of the present methodology.

Fabric article: An article of manufacture which is comprised in whole or in part of fibrous matter matrices or of discrete fibers; and is arranged or fashioned to form a discrete cloth, gauze, cord, or film. The fibrous matter matrices or discrete fibers constituting the tangible substance of the fabric article may be chosen from organic substances, synthetics, prepared polymer compounds, or naturally-occurring materials. In addition, the fabric article may alternatively be prepared as either a woven material or non-woven material, as these different construction forms and formats are conventionally known in the industry and commercially prepared today. Accordingly, a woven fabric may alternately be a textile or knitted article comprised of a single fiber film; or be a single layer or thickness of fibers; or exist as multiple and different deniers of fibers which are present in a range of varying thickness, dimensions, and configurations. Similarly, the non-woven fabric article may comprise single or multiple kinds of fibrous matter as matrices or webs; be prepared by any of the conventionally known processes for making non-wovens; and exist in a wide variety and range of weights, thicknesses, and fluffs.

Antibiotic: An antimicrobial agent or family of agents having a particular chemical formulation and structure which has a demonstrable set of bacteriostatic and/or bacteriocidal, or alternatively, fungiostatic and/or fungiocidal properties against a range of different infectious microbes, including the medically identifiable pathogenic bacteria and/or fungi of a particular order, genus and species. The range of antimicrobial properties (narrow or broad spectrum) and the manner (mechanism of action) by which such antimicrobial properties are characterized, measured, or determined is a matter of conventional knowledge and routine practice in this field. As is described in greater detail hereinafter, the antibiotic of choice employed in the present invention comprises at least one ring structure as part of its composition and formulation. One preferred class of composition comprising such rings structures are the fluoroquinolones (including Ciprofloxacin, Ofloxacin, Norfloxacin, Sparfloxacin, Tomafloxacin, Enofloxacin, Lovafloxacin, Lomefloxacin, Pefloxacin, Fleroxacin, Avefloxin, and DU6859a). Other ringed structure antibiotics such as Doxycycline and Linezolid are additional examples. Similarly, the class of antifungal compositions illustrated by Diflucan can be employed.

Aqueous mixture, liquid or fluid: By definition, any mixture, liquid or fluid which contains or comprises water in any meaningful quantity or degree. Although many other compositions, substances, or materials may exist within the mixture, fluid or liquid in a variety of physical states, the bulk or majority of volume for such fluids is water.

Water-miscible substance. By definition, any composition, compound, material or matter in any physical state (i.e., gaseous, liquid or solid) that is capable of being mixed or combined with water. This term thus includes within its meaning a variety of alternative conditions and physical states for any substance which is capable of: (i) being soluble in any meaningful degree in water or an aqueous blending; (ii) being dispersible in any measurable quantity in water or an aqueous blending (whether or not a colloid is formed); (iii) being able to dissolve in any quantity in water or an aqueous blending (whether or not a homogeneous solution is formed); (iv) being able to be mixed or combined while in a simple, linear, branched, or polymerized condition or while existing in an aggregate, complex, clustered or confluent state; (v) becoming ionized or ionisable in water or an aqueous mixture; and (vi) being able to be distributed in any degree in water or an aqueous mixture while in a non-ionized state or condition.

Woven fabric: A cloth where discrete fibers are first combined into yarns, and the yarns are then interlaced together in some degree during a fabrication process to produce the resulting woven fabric. For purposes of the present invention, any fabric comprised of yarns is by definition is deemed to be a woven fabric, without regard to the particular manner of its combining of fibers into yarns; and is alternatively exemplified by interwoven, knitted, and interlaced yarns as woven fabric articles

Non-woven fabric: A web of material produced directly from fibers without first making yarns. The web of fibers is produced by carding, air-layering or wet-laying; and is subsequently bonded or entangled by needle punching, water-jets (“spunlacing”), chemical glues, or by using chemical means. Those methods that combine web formation and bonding include melt blowing. The non-woven manufacturing process is typically used to yield light-weight, disposable fabrics and cloths.

Fabricated textile: An article of manufacture which is comprised, in whole or in part, of fibers arranged as a fabric; and without regard to whether the fabric is woven or non-woven. The fibers comprising the fabricated textile conduit may be chosen from a diverse range of organic synthetics, prepared polymer compounds, or naturally-occurring matter. In general, the fabricated textile is prepared as a cloth or fabric; and may comprise a single fiber film, or a single layer of fibrous matter, or exist as multiple and different deniers of fibers which are present in a range of varying thickness, dimensions, and configurations.

### I. The Fabric Articles

The method of the present invention is directed to the making of fabric articles useful as wound dressings. This term “fabric article” has been specifically defined above in both meaning and scope; and thus applies to any article, device, appliance, or construct of determinable dimensions and configuration which has as a component part, or contains, or is comprised of a fibrous matter in the form of a matrix or of discrete fibers, in whole or in part.

The broad scope and encompassing coverage of this term “fabric article” is intentional; and, as defined herein, is deemed to cover and apply to any and all non-woven and woven fabrics, cloths, cords, films and other material constructions; includes any and all devices, items, entities, apparatus, appliances, and instruments which present a fabric which is biocompatible with the body of a living subject, be it human or animal. Merely to illustrate some commonly occurring examples, a representative (but incomplete) listing of specific articles is presented by Table 1 below.

TABLE 1
Illustrative And Exemplary Fabric Articles

wound treatment dressings, films, and/or sheets;
gauze pads;
absorbent sponges;
bandages;
prosthetic vascular grafts;
sewing cuffs; and
suture.

Fibrous Matter Existing as Matrices or Discrete Yarns

By definition and practical requirement, the fabric substance of each manufactured article is comprised of fibrous matter prepared as either fiber matrices (or webs), or as woven yarns (or threads). The individual fibers are thus used to form both non-woven and woven fabrics; and may alternatively be composed of a single naturally-occurring matter, or a blending of naturally-occurring substances, or a single synthetic material, or a blending of multiple synthetic compositions, or a mixture of naturally occurring matter and synthetic materials in a wide range of varying ratios.

Merely to illustrate the range and variety of fibrous matter matrices (as used in non-woven articles) and of discrete yarns (as used in woven articles) which are deemed to be suitable for use, the non-exhaustive listings of Tables 2 and 3 are presented below. It will be noted that the listing of Table 2 presents both the natural fibrous matter commonly used for manufactured goods as well as the less commonly used matters and materials existing in nature. In contrast, the listing of Table 3 provides some representative polymeric compositions which are commonly used in industry as well as the less frequently employed synthetic substances which are suitable for use as fibrous matter matrices or discrete yarns.

These substances and prepared compositions can exist in many diverse styles such as knitted or braided textiles; can take form as non-woven layers and deniers of fibrous matter matrices; can appear as fabrics of varying thickness; can be fashioned as material films, sheets, or cloths; and, with any or all of these formats, be combined with other non-fabric components to generate a single construct, unified assembly, or integrated article.

TABLE 2
Illustrative Naturally-Occurring Fibrous Matter And Fibers

Natural Materials/Protein
silk;
wool;
and any mixture of these.
Natural Materials/Cellulose
cotton;
flax or linen;
ramie;
hemp;
paper;
wood;
and any mixture of these.

TABLE 3
Illustrative Synthetic Fibrous Materials And Fibers

Polymeric Compounds And Compositions
polyethylene terephthalate;
nylon;
polyurethane;
polyglycolic acid;
polyamides;
and mixtures of these substances.
Other Synthetic Materials
acetate;
triacetate;
acrylics such as acrylonitile;
aramid;
olefins such as polypropylene and polyethylene;
polytetrafluoroethylene;
polyesters;
saran.

At least some of the formed fibrous matter (matrices or fibers) comprising the fabric article portion of the unique wound dressing (regardless of whether composed of naturally-occurring substance, synthetic materials, or a mixture of these), will demonstrate certain properties and characteristics. Among them are the following:

1. The formed fibrous matter (matrices or yarns formed of fibers) will have a demonstrable capacity to take up water and/or aqueous liquids and fluids (with or without direct wetting of the material). The mode or mechanism of action by which water and aqueous fluids is taken up by the fibrous matter webs or discrete yarns of the fabric (and/or become wetted by the aqueous fluid) is technically insignificant and functionally meaningless. Thus, among the different possibilities of water uptake are the alternatives of: absorption; adsorption; cohesion; adhesion; covalent bonding; non-covalent bonding; hydrogen bonding; miscible envelopment; water molecule entrapment; solution-uptake between matrix/fibers; matrix/fiber wetting; as well as others well documented in the scientific literature. Any and/or all of these may contribute to water or aqueous fluid uptake in whole or in part. Which mechanism of action among these is actively in effect is irrelevant.

2. By choosing the particular chemical formulation and/or stereoscopic structure for the substance of the fibrous matter (matrices or yarns formed of fibers), the selected kind of material as a whole and the resulting fabric may be prepared as articles having either a relatively short life span or a meaningfully long duration for functional use. Thus, by choosing one or more synthetic polymers having recognized water-erosion and biodegradation properties, the fabric article can be manufactured as a biodegradable material with an expected useful life span of only days or weeks. In the alternative, by choosing only durable and highly resilient matter as the fibrous matter (matrices or discrete yarns formed of fibers), fabrics of far longer duration may be routinely made. All of these choices, variables, and alternatives are conventionally known practices commonly available and used by practitioners in this field.

3. The formed fibrous matter (webs or yarns formed of fibers) comprising the fabric article can be utilized in a variety of structures and organizations to form a porous framework or a porous design and pattern. Thus, as conventionally recognized within the industry, the fabric may alternatively be a woven or non-woven construction; may exist either as a single layer fabric or be prepared in multiple layer construct form where each layer may vary in denier size or thickness; and may receive one or more surface treatments, protein coatings, or chemical overlays to import or enhance desired attributes such as in-vivo biocompatibility, a scoured external surface, or greater resiliency over time. All of these porous organizational variances and constructional alternatives are routine matters which will be chosen as a matter of particular needs or personal choices.

4. The formed fibrous matter (webs or yarns formed of fibers) comprising the wound dressing article can be prepared to meet the particular intended use circumstances or contingencies of the particular application. Thus, the constructed fabric can alternatively be prepared as thick cloth or thin gauze; or as a thick-walled configured tube; or as a thin film. Equally important, the resulting construct may take form either as a stiff, inflexible or unyielding length of cloth; or as a very flexible, geometrically configured fabric segment; or even as a cord or string-like length of material.

5. The formed fibrous matter (matrices or yarns formed of fibers) comprising the wound dressing article can be prepared and exist in two markedly different and alternative chemical contexts. Each of these alternatives is as follows.

A first circumstance and context is as fibrous matter comprised of at least one substance or compound which intrinsically provides multiple reactive chemical groups as part of its conventional chemical composition and/or formulation. After a discrete fibrous matter article has been formed, these native and built-in multiple reactive chemical groups appear at and are a normal part of the original, unmodified exterior surface. These reactive groups remain available and functional for subsequent chemical reaction after the fibrous matter has been formed as a discrete article; are often present in more than one type or species (such as amine, carboxyl, and hydroxyl groups); and typically will steroscopically exist at as well as extend from the unmodified exterior surface of the fibrous matter into the ambient environment.

A second circumstance and context is as fibrous matter comprised of at least one substance or compound which is substantially devoid of functional reactive chemical groups as part of its conventional chemical composition and/or formulation. In this second context, the exterior surface of the formed fibrous matter must be chemically modified using a diverse variety of chemical agents to create multiple reactive groups at the exterior surface of the formed fibrous matter. Accordingly, in this instance, a plurality of reactive chemical groups is synthetically or extraneously obtained and intentionally joined to the fibrous matter as a necessary chemical modification of the original exterior surface; and, after being obtained and so positioned, these extraneous or synthetically generated reactive chemical groups will then remain functional at the exterior surface for subsequent chemical reaction with other molecules of interest.

### II. The Antibiotics of Choice

### The Structural and Chemical Similarities Between Some Textile Dyes and Certain Antibiotics

Dyes (organic compounds that are colored) must possess certain properties for binding such as demonstrable solubility during application; a degree of fibrophilicity; and fastness for selective fibers. The number of chemical structures that possess such properties is extensive, with several thousand dyes commercialized.

Most dyes are based on azo- and anthraquinone chemistry, although many other chemical types have been used. A majority of these dyes have relative molecular masses (r.m.m.) in the 300-1,500 range; and depending on the fiber to which they are applied, can be anionic (usually via sulfonic or carboxylic acid groups), cationic (quaternized nitrogen) or nonionic with slight solubility derived from hydrophilic hydroxy or amino groups. Beyond the r.m.m. and functionality of the selected dye, it is difficult to predict the extent or strength of interaction between dye and fiber based solely on molecular structure. Disperse dyes, a class of dyes that have a strong affinity for polyester, are of particular interest and represent the type of interaction that would be a model for assessing antibiotic adhesion to the surface.

Similar to dyes, there are many types of compounds that have antimicrobial activity. Antibiotics have numerous functions, from prevention of bacterial wall formation to inhibition of DNA function and protein synthesis. Their mode of action is directly dependent on their detailed chemical structure, which can vary widely between different classes of antibiotics but can vary slightly within the same class. These variations in structure distinguish the various families of antibiotics, spectrum of activity, side effects, and clinical usefulness. Also, many antibiotics have structural features (solubility, r.m.m., anionic or H-bond forming functional groups) that are comparable with those of dyes.

### The Releasable Antibiotics of Choice

The Fluoroquinolone Antibiotics

In order to utilize antibiotic using dyeing conditions, the antibiotic structure must have a relative molecular mass (r.m.m.) in the 300-1,500 range as well as be a heat stable composition. A “compact” chemical structure, based on aromatic rings of disperse dyes, would also be a requirement.

The exemplary fluoroquinolones are of particular interest and value. This family of antibiotics now includes at least twelve members (Ciprofloxacin, Ofloxacin, Norfloxacin, Sparfloxacin, Tomafloxacin, Enofloxacin, Lovafloxacin, Lomefloxacin, Pefloxacin, Fleroxacin, Avefloxin, and DU6859a); and the fluoroquinolone family as a whole has become the drug of choice for many applications. These antibiotics are effective at low concentrations; and hold an ideal antimicrobial spectrum against microorganisms most commonly encountered clinically in wound infection, with significant activity against many relevant pathogens—such as S. aureus, methicillin-resistant S. aureus, S. epidermidis, Pseudomonas species, and Escherichia coli. Moreover, Fluoroquinolones are heat stable; are of 300-400 r.m.m.; and have many structural features analogous to dyes. Accordingly, this family of antibiotics possesses those characteristics which are highly desired for fabricated textile use.

### The Manner for Incorporating a Releasable Antibiotic onto Fibrous Matter

The Pad/Heating (or Pad/Dying) process, is conventionally known as a high temperature dyeing technique that opens the fibrous matter structure of wovens and non-wovens [see Hoechst Celanese, Dictionary of Fiber & Textile Technology, 1990]. The conventional Pad/Heating process was therefore evaluated and tested as an alternative procedure with a chemically modified fibrous matter such as polyester. The result of this process yielded fibrous matter articles whose exterior surface demonstrated substantial antimicrobial activity for a period of time greater than 50 days in duration.

Nevertheless, while the high temperature Pad/Heating technique was clearly successful for incorporating Cipro into the surface of the prepared fibrous matter, the elevated temperatures required for the Pad/Heating processing were not conducive for maintaining the integrity of the chemical groups within the fibrous material. For this reason, an entirely new and much improved technique, now termed the “Pad/autoclaving” process, was developed; and this unique and newly developed technique is presently the method of choice for incorporation of an antibiotic, such as Cipro, onto the exterior surface of a fibrous matter article.

### The Releasable Antibiotic Reaction Product

The antibiotic of choice is to be temporarily-incorporated onto the fibrous matter, preferably via the Pad/Autoclave technique. However, there several minimal chemical requirements and functional qualifications which the antimicrobial must provide in order to be suitable for use in the present invention. These are:
(1) The antibiotic of choice must be capable of being temporarily captured and incorporated as a discrete moiety into said fibrous matter;
(2) The antibiotic composition must demonstrably retain its characteristic biological activity after being temporarily immobilized and held upon the fibrous material; and
(3) The antibiotic moiety must demonstrably retain its characteristic biological activity after being released from the fibrous matter surface and becoming a freely mobile antibiotic molecule in-situ.

### III. The Proteins Which can be Effectively Covalently Bound at the Fibrous Matter Surface in Tandem with an Incorporated Antibiotic

### The Manner of Indirectly Covalently Binding a Biologically-Active Protein

A. When making the wound dressing of the present invention, the antibiotic of choice is presumed to have been captured, held and temporarily immobilized upon the exposed surface(s) of the formed fibrous matter in the dry state, as described herein. What then remains to be done, therefore, is to bind at least one biologically active protein covalently to the exterior surface in tandem with the previously incorporated antibiotic.

For this goal to be accomplished in a reliable and reproducible manner, the intrinsic chemical properties of the formed fibrous matter must again be taken into account. It will be recalled that the formed fibrous matter (webs or yarns formed of fibers) comprising the wound dressing article can be prepared and exist in two markedly different and alternative chemical contexts: The first circumstance and context is when the fibrous matter is comprised of at least one substance or compound which intrinsically provides multiple reactive chemical groups as part of its conventional chemical composition and/or formulation. The native and built-in multiple reactive chemical groups appear at and are a normal part of the unmodified exterior surface; and these reactive groups remain available and functional for subsequent chemical reaction after the fibrous matter has been formed as a discrete article.

The second circumstance and context is when the fibrous matter is comprised of at least one substance or compound which is substantially devoid of functional reactive chemical groups as part of its conventional chemical composition and/or formulation. In this second context, therefore, the exterior surface of the formed fibrous matter must be chemically modified using at least one chemical agent able to create and provide multiple reactive groups as anchorage sites on the exterior surface of the formed fibrous matter; and, as a consequence, a plurality of reactive chemical groups must be extraneously or synthetically obtained and intentionally joined to the fibrous matter as a necessary chemical modification of the exterior surface. Afterwards, these intentionally generated reactive chemical groups will remain functional at the exterior surface for subsequent chemical reaction.

B. In the overwhelming majority of instances, it is expected that the fibrous matter of the wound dressing will be of the second circumstance and context; and that a plurality of reactive chemical groups must be obtained and chemically attached to the fibrous matter as a chemical modification of the exterior surface. The preparation of the preferred embodiments, including the empirical examples provided hereinafter, fall into this second category where a chemical modification of the exterior surface becomes necessary in order to later bind a biologically active protein of interest.

To achieve this purpose, a diamine coupling agent is desirably employed to create and provide a plurality of different functional reactive groups (such as amine groups and carboxylic acid groups) upon the exposed surfaces of the fabricated textile. One preferred coupling agent is ethylenediamine.

It will also be noted that among the useful diamine coupling agents are those having at least one amine group available for subsequent binding. These agents chemically constitute merely one recognized family of compounds, all which are conventionally known and commercially available. Among them, are those substances exemplified and listed by Table 4 below.

TABLE 4
Representative Diamine Coupling Agents

PolyethyleniminesMn 400 to 10,000;
(Polypropyleneglycol) bis(2-Aminopropyl ether) Mn 200 to 4000;
Ethylenediamine;
1,3 Propylenediamine;
1,2 Propylenediamine;
Neopentadiamine;
Butylenediamine;
Pentylenediamine;
Hexamethylenediamine;
Octamethylenediamine;
Diethylenetriamine;
N-(2-Aminopropyl)-1,3-propanediamine;
N-(3-Aminopropyl)-1,3-propanediamine;
N,N″-1,2-Ethylene bis(1,3-propanediamine) Tetraethylenepentamine.

C. After chemically modifying the exposed surfaces of the fabricated textile to create and provide the plurality of functional reactive groups (such as amine groups and carboxylic acid groups), these reactive groups then serve as a plurality of anchorage sites for the subsequent attachment of one or more biologically active proteins to the exterior surface of the formed fibrous matter.

However, chemical juncture to these multiple anchorage sites—the functional reactive groups then existing on the modified surface of the fibrous matter—is preferably made by using one or more bifunctional linking agents. Via this indirect manner of juncture, the permanent immobilization and covalent binding of a biologically-active protein (such as thrombin) to the modified exterior surface of the preformed fibrous matter is achieved, and exists in tandem with a previously incorporated, but later releasable, antibiotic.

### The Choices of Biologically Active Protein

A number of different biologically active proteins can be beneficially and advantageously utilized in tandem with the antibiotic of choice when constructing and chemically preparing the surface of the fibrous matter as a wound dressing. However, there several minimal chemical requirements and functional qualifications which the protein must provide in order to be suitable for use in the present invention. These are:
(i) The protein must demonstrably retain its characteristic biological activity both before and after being covalently bound to the exterior surface of the fibrous matter;
(ii) The covalently bound protein must demonstrably retain its characteristic biological activity both before the incorporated antibiotic is released from the fibrous matter surface and after the antibiotic is released and becomes a freely mobile molecule; and
(iii) The protein covalently bound to the bound to the exterior surface of the fibrous matter must demonstrate its characteristic biological activity in-situ concurrently with the mobile antibiotic's antimicrobial activity in-situ.

In addition, since the primary application of the fibrous matter in the fabricated article is as a wound dressing, it is preferred that the biological properties and characteristics of the chosen protein serve as an adjunct to and promoter/enhancer of the natural healing processes occurring in-vivo for the repair and/or reconstruction of human body tissues. Accordingly, it is deemed most desirable that the primary function and biological capabilities of the chosen protein not be anti-microbial as such; but rather that the chosen protein act selectively as a physiological aid and/or pharmacological assistant to promote blood coagulation, or to act as a chemotactic factor which attracts or activates specific types of local cells in-vivo, and/or function to augment or to mediate the variety of different humoral and cellular responses associated with or related to inflammations and the inflammatory response as well as the immune response in-vivo.

Merely to illustrate the diverse range and variety, as well as to provide some representative examples of commonly available choices, a non-exhaustive listing of some proteins preferred for use with the present invention is presented by Table 5 below.

TABLE 5
Representative Proteins Which Retain Biological Activity
After Being Covalently Bound
Blood coagulation cascade proteins

Prothrombin;
Thrombin;
Fibrinogen;
Factors IV, V, VIII, and X individually.

Selected Cytokines
Interleukin-1 (IL-1), an endogenous pyrogen and major inflammatory mediator;
Interleukin-2 (IL-2), a T-cell activator and growth factor;
Interleukin-3 (IL-3), a hematopoietic growth factor;
Interleukin-4 (IL-4), a T-cell and B-cell growth factor;
Interleukin-5 (IL-5), a promoter of eosinophil growth and differentiation and IgA antibody synthesis;
Interleukin-6 (IL-6), a B-cell differentiation factor;
Interleukin-7 (IL-7), a growth factor for early B- and T-lymphocytes;
Interleukin-8 (IL-8), a chemotactic factor for neutrophils and lymphocytes;
Interleukin-10 (IL-10), a down-regulator of cell activation;
Interleukin-12 (IL-12), an augmenter of IFN-γ production;
Interleukin-13 (IL-13), a factor which overlaps in function with IL-4;
Tumor necrotic factor (TNF), a factor which overlaps in function with IL-1 and mediates host response to gram-negative bacteria;
Interferons-α, -β, -γ, which activate macrophages, enhance lymphocyte and natural killer cells, and have antiviral and antitumor activity;
Granulocyte-macrophage colony stimulating factor (GM-CSF), a growth factor for granulocytes, macrophages, and eosinophils.
   Growth Factors
Fibroblast Growth Factor;
Vascular Endothelial Growth Factor;
Platelet Derived Growth Factor;
Bone Morphologic Protein (a family of compositions).

### The Means and Manner of Protein Attachment

The protein of choice is to become covalently or ionically bound via a bifunctional linking molecule/agent to the chemical reactive group(s) present on the exterior surface of the fabric article. Afterwards, when the protein has become permanently bound via the bifunctional linking molecule/agent and is integrally attached to the surface of the fibrous matter, the manufacturing process will be complete; and a ready to use wound dressing will be the result.

The Bifunctional Linking Molecule

The term “bifunctional linking molecule (or agent)” is defined herein as a crosslinking composition or chemical agent having the ability to bind to two reactive groups or moieties found on either the same entity or on different entities. The bifunctional linking molecule will thus serve to connect these two reactive groups sterochemically; and, as an intermediate, join the two reactive groups together as an unified chemical structure.

By definition, a heterobifunctional linking molecule or agent is one that binds to two different types of reactive groups and joins them together as a unified structure. Conversely, if the bifunctional linking molecule binds two similar or identical reactive groups, it is referred to as a homobifunctional linking molecule or agent.

A wide range and variety of heterobifunctional and homobifunctional linking molecules are conventionally known in the scientific literature and are commercially available. Thus, some representative heterobifunctional linking molecules or compounds suitable for use in the instant methodology include, but are not limited to: sulfosuccinimidyl 4-(N-maleimidomethyl) cyclohexane-1-carboxylate (sulfo-SMCC); succinimidyl 4-(N-maleimidomethyl) cyclohexane-1-carboxylate (SMCC); Nsuccinimidyl-3-(2-pyridyldithio) propionate (SPDP); sulfosuccinimidyl 2-(7-azido-4-methylcoumarin-3-acetamide) ethyl-1,3′-dithiopropionate (SAED); 1-ethyl-3-(dimethylaminopropyl)-carbodiimide HCl (EDC); and Traut's reagent (2-iminothiolane hydrochloride).

In addition, a diverse choice of homobifunctional linking molecules or compounds can also be usefully employed in this methodology and include, but are not limited to: ABH; ANB-NOS; APDP; APG; ASIB; ASBA; BASED; BS³; BMH; BSOCOES; DFDNB; DMA; DMP; DMS; DPDPB; DSG; DSP; DSS; DST; DTBP; DTSSP; EDC; EGS; GMGS; HSAB; LC-SPDP; MBS; M₂C₂H; MPBM; NHS-ASA; PDPH; PNP-DTP; SADP; SAED; SAND; SANPAH; SASD; SDBP; SIAB; SMCC; SMBP; SMPT; SPDP; Sulfo-BSOCOES; Sulfo-DST; Sulfo-EGS; Sulfo-GMBS; Sulfo-HSAB; Sulfo-LC-SPDP; Sulfo-MBS; Sulfo-NHS-ASA; Sulfo-NHS-LC-ASA; Sulfo-SADP; Sulfo-SAMCA; Sulfo-SANPAH; Sulfo-SAPB; Sulfo-SIAB; Sulfo-SMCC; Sulfo-SMBP; and Sulfo-LC-SMPT.

The chosen bifunctional linking molecule or compound is first covalently reacted with and joined to the pre-existing pendant amino group(s) chemically created or naturally-occurring within the fibrous matter, which are functionally available for chemical reaction; and second, reacted subsequently with at least one biologically active protein in a quantitative amount such that the desired degree of binding site density is achieved with the active protein of choice. The preferred binding site density is provided by that amount of bifunctional linking molecules that optimizes the cross-linking reaction coverage of that surface for the subsequent covalent binding and juncture of the active protein of choice. The intended consequence and result of these bifunctional linking reactions is the covalent juncture and steroscopic immobilization of the chosen biologically active protein to the material surface; and the formation of a biologically activate surface for the fibrous matter comprising the fabricated article

The Purpose and Manner of Creating Multiple Bifunctional Reactive Groups

The chemical groups positioned on and extending from the exterior surface of the fibrous matter will exist either in a natural fashion owing to the nature of the chemical composition for the fibrous matter itself; or are intentionally created on the exterior surface of the fibrous matter by chemical intervention.

The latter technique intends that bifunctional linking molecules become reactively joined to the multiple functional reactive groups serving as anchorage sites on the exterior surface of the fibrous matter; and via such attachment to the surface of the fibrous matter, provide for cross-linking and covalently binding the biologically active protein of choice to the fibrous matter—in a manner which is independent of and is concurrent with the previously incorporated antibiotic.

Clearly, the bifunctional reactive groups created on the exterior surface of the fibrous matter serve as cross-linking agents and chemical bridges which link the multiple functional reactive groups serving as anchorage sites on the exterior surface of the fibrous matter to the protein of choice, without interfering with either the antimicrobial activity or subsequent releasability of the incorporated antibiotic, then also present at the surface of the fibrous matter. The resulting fibrous matter surface thus has both active protein capabilities as well as antimicrobial properties.

The preferred mode for permanently attaching the active protein of choice is via the use of at least one, and desirably two, bifunctional linking agents. Such bifunctional linking agents are well recognized and conventionally known as a class of chemical compositions which function as cross-linking binders; and are routinely employed (either singly or in multiples) to join one kind of molecule to another. The specific choices of use concentration ranges, order of reactants, reactions times and conditions, and the like are conventionally known variable; and are thus left to the personal needs or preferences of the user.

The Expected Order of Linking Reactions

It is typically expected that when an active protein of choice and a bifunctional linking molecule are reactively combined together, an identifiable intermediate entity or coupled complex will be generated as a consequence. The intermediate entity is in fact a complex form of the bifunctional linking molecule joining with the protein of choice to form a discrete complex. However, because a bifunctional linking molecule is employed as the reactive moiety, the generated intermediate entity and complex will retain at least one unbound and functionally reactive group for another subsequent chemical reaction; and, in this manner, the intermediate entity can subsequently become covalently or ionically bound to the immobilized antibiotic moiety then present on the surface of the fabric article.

Accordingly, as the empirical evidence presented hereinafter by the illustrative example clearly shows, when thrombin (the protein of choice) and Sulfo-SMCC (the 1^{st} bifunctional linking agent) are reactively combined together, a thrombin-SMCC intermediate entity and complex is generated as the result.

Then, when the generated thrombin-SMCC intermediate complex subsequently is reactively added to the antibiotic immobilized fabric article prepared in Traut's reagent (the 2^{nd} bifunctional linking agent), a second binding reaction occurs; and a permanent immobilization and binding of the thrombin to the antibiotic molecule then immobilized on the surface of the fabric article is the result. In this manner, via the preferred use of multiple bifunctional linking agents to attach the protein of choice to the antibiotic molecule, a wound dressing having a biologically active antibiotic/protein complex on its fabric surface is obtained.

### IV. The Intended Clinical Applications for the Wound Dressing

The kinds of clinical applications for the unique wound dressing is intended to include major traumatic wounds caused by accident, negligence, or battlefield conditions; planned surgical incisions and invasive body surgical procedures performed under aseptic conditions; transcutaneous incisions and vascular openings for catheter insertion and blood vessel catheterization procedures; and other body penetrations and openings made for therapeutic and/or prophylactic purposes.

The wound dressings provided by the present invention thus are intended and expected to be manufactured as pre-packaged and pre-sterilized fabric articles; be an item which can be prepared in advance, be stocked in multiples, and be stored indefinitely in a dry state without meaningful loss of biological function or efficacy; and serve effectively as an antimicrobial and healing dressing for the various kinds of wounds as they exist under the many different clinical circumstances and medical conditions where a penetration of the body has occurred.

The wound dressing fabric articles should be manufactured and tailored in advance to meet a wide range of intended use circumstances or contingencies expected to be encountered in a particular situation. For this reason, the constructed fabric article can and should alternatively be prepared as a thick cloth and as a thin gauze; as a solid-walled configured tube and as a delicate film. Equally important, the resulting construct may take physical form either as a stiff, inflexible and unyielding mass or as a very flexible and supple layer; have a varied set of dimensions and girth; appear as both a geometrically symmetrical or asymmetrical configured fabric; and can exist even as a slender cord or string-like length of material.

Medically, the wound dressing fabric articles of the present invention can be employed in-vivo in the following ways: topically or subtopically; transcutaneously, percutaneously, or subcutaneously; or internally within the body's interior; vascularly or humorally; and applied to any kind of body cavity, body tissue or body organ without regard to anatomic site or location.

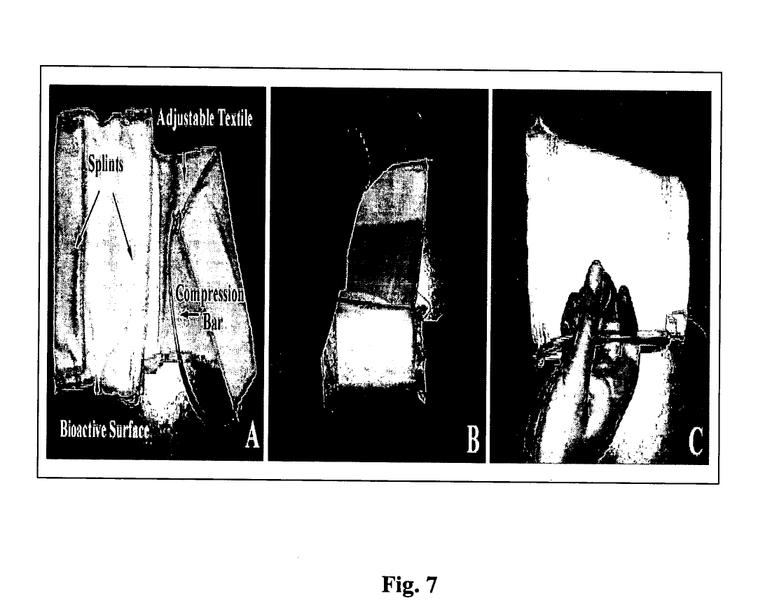

The wound dressing fabric articles of the present invention are also intended to serve as compression dressings. One embodiment of this novel lightweight wound dressing prototype is illustrated by FIG. 7A. As shown therein, the chosen format and design of the wound dressing fabric article is such that the prepared surface of the fibrous matter can be rapidly applied and adjusted by a trained healthcare provider (e.g., an emergency medical technician, a firefighter, or an emergency room nurse or physician); and/or can be applied to a traumatic wound by the injured person himself if he is not able to obtain immediate medical assistance from others (e.g., outdoorsman in the field such as injured hunters and fishermen, and military personnel such as wounded soldiers under battle field conditions). The wound dressing article and prepared surface can be applied with compression force, and later be manually adjusted (as well as readjusted) at will or as medically necessary by either the trained medical attendant or the injured person himself (see for example, FIGS. 7B and 7C respectively).

### V. A Preferred Method for Making a Wound Dressing Article

### Obtaining a Preferred Dacron-Polyurethane Fabric

One preferred type of fibrous matter is termed “D-PU material”, which is composed of 80% Dacron and 20% polyurethane. “D-PU material” is a fiber-base, woven textile which can be commercially obtained in bulk from Darlington Fabrics (Westerly, R.I.); and is a knitted fibrous matter in which the polyurethane component is inlayed into the knitted structure in the weft direction. D-PU material is commercially sold as knitted sheets of fabric (i.e., a cloth); and a wide range of differently configured and dimension-sized cloth segments of fibrous matter can be cut from a single large knitted sheet of fabric.

After being cut to the preferred size dimensions and desired configuration, the individual fabric segments are individually washed in a scouring solution (composed of 10 g Na₂CO₃ and 10 ml Tween 20 in 1 liter of double distilled water) for 30 minutes at 60° C. The scoured segments are then rinsed in double distilled water for an additional 30 minutes at 60° C., and then air-dried at room temperature (about 20-25° C.) overnight.

### A Fabric Surface with Accessible Bifunctional Groups

The preferred creation of a bifunctionalized surface for the D-PU fabric segments is achieved by use of a preferred diamine agent, ethylenediamine (hereinafter “EDA”). However, any of the other diamine agents listed by Table 4 herein will also serve well for this purpose.

Preferably, the washed D-PU sized segments are placed in 100% EDA for 80 minutes at environmental room temperature (about 25° C.). If desired however, EDA incubation times ranging from about 10-120 minutes may be employed to achieve amine/carboxylic acid formation upon varying exposure to the bifunctional amine. The bifunctionalized sized fabric segments (now identified as “bD-PU”) are then removed and placed into distilled water overnight at environmental room temperature (about 25° C.).

When using the preferred diamine agent, ethylenediamine (“EDA”), it will be noted that the degree of amination and carboxylation resulting on the fabric surface will vary in part as a function of EDA reaction time with the Dacron surface of the bD-PU sized segments. A shorter reaction period (a time meaningfully less than about 80 minutes) yields a fabric surface having fewer functional groups. However, it was found that a longer reaction period well beyond about 85-90 minutes duration significantly alters the strength of the materials; and does not meaningfully add to the total number of bifunctional groups created within the Dacron polymer than that number achieved by a shorter reaction time of only 80-90 minutes duration.

Also, the polyurethane fibers existing within the blended knit structure are not affected by prolonged exposure to EDA since no dye uptake occurred (data not shown). Macroscopically, the bifunctional bD-PU material possessed comparable stretch to the original D-PU material.

### Application of Antibiotic onto bD-PU Segments Using Textile Dyeing Technology

An Unacceptable Technique

Textile dyeing techniques are preferably employed to incorporate a broad-spectrum fluoroquinolone antibiotic such as Ciprofloxacin (hereinafter “Cipro”) into the bD-PU material. Previous studies using unmodified Dacron fiber have revealed that Cipro uptake into the chemically unmodified fibers was unsuccessful using the conventional solution dyeing procedure; and also showed that after extensive washing of the fabric article, such antimicrobial activity as was then present lasted less than 4 hours' time. This very limited affinity of Cipro for the unmodified Dacron fiber is believed to be the result of the limited hydrophilic properties possessed by the Dacron fiber in its original, chemically unmodified, form.

An Acceptable Technique

The Pad/Heating (or Pad/Dying) process, is conventionally known as a high temperature dyeing technique that opens the fibrous matter structure of wovens and non-wovens [see Hoechst Celanese, Dictionary of Fiber & Textile Technology, 1990]. The conventional Pad/Heating process was therefore evaluated and tested as an alternative procedure with the chemically modified Dacron fiber having bifunctional groups, the bD-PU segments (prepared as described above); and the result of this process yielded fibrous matter articles whose exterior surface demonstrated substantial antimicrobial activity for a duration of time greater than 50 days.

Nevertheless, while the high temperature Pad/Heating technique was clearly successful for incorporating Cipro into the surface of the prepared bD-PU segments, the elevated temperatures required for the Pad/Heating processing were not conducive for maintaining the integrity of the ethylenediamine (EDA) reactive groups then already attached to the material surface of the fibrous matter. For this reason, and since EDA exposure creates a more hydrophilic surface (via the presence of amine and carboxylic acid reaction groups) on the material surface, a new and much improved technique, now termed the “Pad/autoclaving” process, was developed; and this unique and newly developed technique is presently the method of choice for incorporation of an antibiotic such as Cipro onto the prepared exterior surface of a fibrous matter article.

The Preferred New “Pad/Autoclaving” Process

The knitted and dimension-sized bD-PU segments prepared as described above are employed as the fabric articles comprised of fibrous matter. The bD-PU segments are then placed into a Cipro-containing dyebath that was prepared as follows: liquor ratio (ratio of water weight to material weight)=20:1; percent antibiotic on the weighed fabric (“% owf”)=5%; bath pH=8.0; the preferred dyeing time=2 hours; and the preferred dyeing bath temperature=70° C.

As an illustrative and preferred technique, for 1 g of bD-PU segment, 50 mg of Cipro was dispersed in 20 ml water (or another aqueous based liquid of interest). After dyeing in the bath liquid for 2 hours at 70° C. and air-drying overnight at environmental room temperature, the Cipro-dyed segments (now termed “bD-PU-AB”) were then autoclaved (i.e., steam heat at 130° C.-200° C.) for 15 minutes under pressure, followed by a 10 minute drying time at environmental room temperature.

Macroscopically, a yellowish hue was visibly evident after the Cipro bath-dyeing and subsequent autoclaving of the bD-PU segments. In comparison, the bD-PU segments placed into the Cipro dyeing bath but not subsequently autoclaved had no gross color change. Also, exposure of pre- and post-dyed bD-PU segments showed no visible difference in Cipro uptake (data not shown), demonstrating that the functional groups on the material surface which were utilized for binding of the antibiotic were not affected by the lower (70° C.) bath temperature used for Cipro attachment.

Also, in so far as is presently known, the requisite steps of the process can be varied in major degree. Thus, for each gram weight of prepared bD-PU segment, any quantity of the antibiotic of choice (such as Cipro) can be dispersed in almost any amount of water (or another aqueous based liquid) in ratios (w/v) so long as an effective aqueous concentration of the chosen antibiotic exists within the liquid mixture. Similarly, the reaction duration for the segments in the bath liquid is selected as a function of time and bath temperature. Thus, while the preferred technique uses 2 hours dyeing time and a bath temperature of 70° C., it is believed that at least 30 minutes reaction time will be needed to be effective, and that a reaction temperature above 20° C. and below 100° C. can be effectively employed. Similarly, after the dyed segments have been air-dried overnight at room temperature, the antibiotic attached segments must be then autoclaved using a steam heat temperature not less than about 130° C.-200° C. under pressure for a short duration less than 30 minutes, followed by a drying preferably at room temperature.

### Covalent Bonding and Permanent Juncture of Thrombin Independently onto the Cipro-Dyed bD-PU Segments

It will be appreciated that thrombin is employed as a preferred and exemplary protein which is to be independently covalently bound to the exterior surface of the fibrous matter of the fabric articles. Accordingly, the bD-PU-AB segments (prepared as described above) are placed in a prepared buffering solution. For this buffering purpose, a stock 50 mM sodium bicarbonate buffer solution (pH 8.5) is preferably prepared as follows: A 20 mg/ml solution of Traut's reagent is prepared in the bicarbonate buffer; and the buffering solution is added to the bD-PU-AB segments in borosilicate glass containers. All the prepared segments are then placed in the buffer solution for a reaction time of one (1) hour at room temperature, using an orbital shaker set at 120 rpm.

Within 20 minutes of completion, a 45 μM thrombin solution (10% thrombin) is then prepared. Sulfo-SMCC (1 mg/ml; 71 μl) is added to the thrombin solution and reacted for 20 minutes at 37° C. in a water bath. The thrombin-SMCC intermediate is then purified via gel filtration (PD-10 fast desalting column) and the peak fractions pooled. The pooled thrombin-SMCC solution is desirably diluted to a final use concentration of 13 μM.

Once the Traut's reaction is complete, all the bD-PU-AB segments are washed twice on the inversion mixer with 5 ml bicarbonate buffer. The thrombin-SMCC solution is then added and allowed to react for about 3 hours duration at room temperature on an orbital shaker (150 r.p.m.). After this incubation time, the sized segments are removed and then washed four separate times in a prepared wash mixture of 0.01M sodium phosphate, 0.5M NaCl, 0.05% Tween 20, pH 7.4 buffer, for 5 minutes on inversion mixer. The wash buffer is changed between individual washes.

The incubation of the bD-PU-AB segments with Traut's reagent results in a markedly greater thrombin binding as compared to bD-PU-AB segments incubated with only bicarbonate buffer. Sonication is typically utilized to remove non-specific protein binding. However, a combination of the wash buffer and sonication was shown to elute Cipro from the material (data not shown). Therefore, only detergent washing of the segments should be employed.

### D. The Unique Wound Dressing Article

The preferred manufacturing procedure described herein results in a biocompatible fabric article whose prepared bD-PU-AB-thrombin surface allows for the slow release of multiple discrete Cipro molecules on-demand-after being placed in an aqueous locale or liquid use environment; offers an pharmacologically active wound dressing biomaterial having demonstrable antimicrobial activity; and includes a covalently bound and permanently immobilized, functional thrombin moiety which is biologically-active despite being joined to the fibrous matter of the fabric article.

### VI. Exemplary Experiments, Results, and Empirical Data

To demonstrate the merits and value of the present invention, a series of planned experiments and empirical data are presented below. It will be expressly understood, however, that the experiments described herein and the results provided below are merely the best evidence of the subject matter as a whole which is the present invention; and that the empirical data, while limited in content, is only illustrative of the scope of the present invention as envisioned and claimed.

### Experimental Series 1: The Preparation Method as a Series of Stages

### Stage A: Creating the bD-PU Material

The Initial Fibrous Matter Composition

The original fibrous matter was D-PU material, composed of 80% Dacron and 20% polyurethane; and was obtained in bulk from Darlington Fabrics (Westerly, R.I.). As a composition of matter, the polyurethane in the original D-PU material is inlayed into the knitted structure in the weft direction.

D-PU segments (5 cm×5 cm in size) were cut from a large knitted fabric sample. The D-PU segments were washed in 500 ml of scouring solution (10 g Na₂CO₃, 10 ml Tween 20 in 1 L double distilled water (ddH₂O)) for 30 minutes at 60° C.; and the scoured samples were then rinsed in 500 ml ddH2O for 30 minutes at 60° C. and air-dried overnight.

The creation of a bifunctionalized surface for the sized segments was achieved by incubation of the washed D-PU segments in 100% ethylenediamine (EDA) for 80 minutes at room temperature. Additionally, EDA incubation times ranging from 10-120 minutes was also evaluated to determine amine/carboxylic acid formation upon varying exposure to the bifunctional amine. The segments were then removed and placed into distilled water overnight at room temperature (bD-PU).

Surface Characterization of the bD-PU Segments

Methylene Blue (MB, 80% purity), a cationic dye, was employed to qualitatively determine the existence of carboxylic acid groups within the bD-PU segments. Briefly, a working solution of MB (5 mg/L) was prepared in 0.1 M Tris-Cl, pH 8.0. Smaller segments (1 cm² in area) were then cut from the bD-PU material. Working MB solution (10 ml) was added to each smaller segment, and the reaction mixture incubated for 1 hour. The reacted segments were then removed and placed into a Tris wash solution for an additional 1 hour's time.

The bD-PU segments were then grossly observed for color uptake and shade differences and then photographed. For amine content, CI Acid Red 1 (AR1), an anionic dye, was employed to qualitatively assess total (primary and secondary) amine content in the bD-PU segments. Briefly, a working AR1 solution (50 mg/L, dye purity=60%) was prepared in 10 mM MES, pH 4.5 (MES). Segments (1.0 cm²) were cut from the respective treatments. Working AR1 solution (3 ml) was added to each segment and incubated for 1 hour. The segments were removed and placed into a MES wash solution for one hour. Segments were then grossly observed for color uptake and shade differences, followed by image photography.

Results:

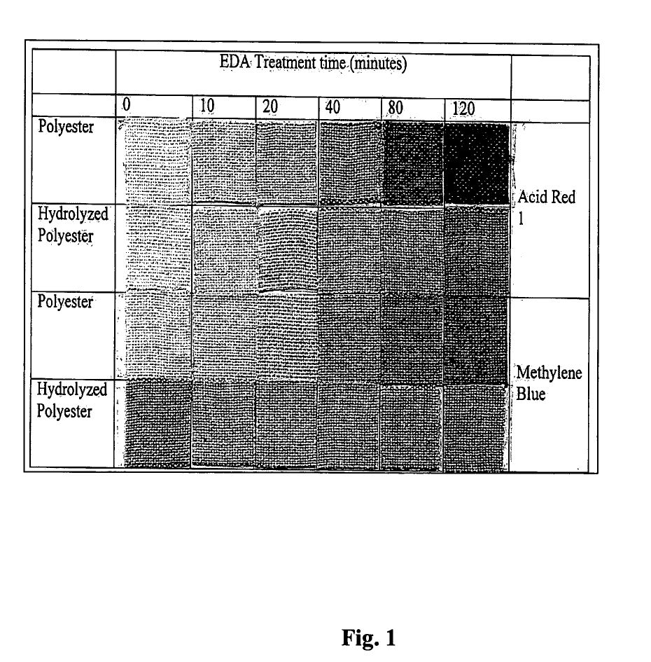

Exposure of the D-PU material to EDA created both carboxylic acid and amine groups within the polymer structure as evidenced by uptake of both MB and AR1 (see FIG. 1). In contrast, the control D-PU material had minimal uptake in either dye solution.

Amination did occur as indicated by AR1 uptake, which is specific for detecting amine formation. No AR1 uptake occurred on sodium hydroxide hydrolyzed surfaces (which creates only carboxylic acid groups), a technique that results in a hydrophilic surface. Thus, amination was confirmed via AR1 uptake.

The degree of amination/carboxylation was also varied upon alteration of EDA reaction time with the Dacron surface, with fewer functional groups created at earlier time periods. The polyurethane fibers within the knit structure were not affected by exposure to EDA since no dye uptake occurred (data not shown). Macroscopically, the bD-PU material possessed comparable stretch to the original D-PU material. This study demonstrated that the D-PU material could be chemically modified, resulting in formation of two (2) independently accessible functional groups. The next step was to determine if Cipro, the antibiotic of choice, could be dyed into the bD-PU material while maintaining the integrity of the EDA functional groups within the polymer structure.

### Stage B. Introduction of a Releasable Antibiotic onto the bD-PU Material

Methods

Various modes of dyeing were then performed in order to incorporate the broad-spectrum fluoroquinolone antibiotic Ciprofloxacin (Cipro) into the bD-PU material. In previous studies using unmodified Dacron, Cipro uptake into the fibers was unsuccessful using solution dyeing, with antimicrobial activity lasting less than 4 hours after extensive washing. The limited affinity of Cipro for Dacron is believed to be the result of the hydrophobic properties of the material.

Pad/Heating, which is a high temperature dyeing technique that opens the fiber structure, was then utilized. This technique demonstrated antimicrobial activity for greater than 50 days. Nevertheless, while this technique was successful for incorporating Cipro, the elevated temperatures demanded by the processing were not conducive to maintaining the bifunctional groups created previously on the material surface.

Since EDA exposure creates a more hydrophilic surface via creation of amine and carboxylic acid groups as indicated by AR1/MB uptake, a modified pad/heat dyeing technique (pad/autoclaving) was examined. Knitted bD-PU segments (3 cm×5 cm) were prepared (as outlined above). The bD-PU segments were then placed into a Cipro “dyebath” that was set up as follows: liquor ratio (ratio of water weight to material weight)=20:1, percent antibiotic of the weighed fabric (% owf)=5%, pH=8.0, dyeing time=2 hours and dyeing temperature=70 C. For example, for 1 g of material, 50 mg of Cipro was prepared in 20 ml water or solution of interest.

After air-drying overnight, the Cipro-dyed segments (bD-PU-AB material) were autoclaved for 15 minutes (10 minute dry). Control segments were treated in a similar fashion, however, no heating or autoclaving were performed. Segments were then washed for 5 days to determine initial antimicrobial activity. Other bD-PU-AB material segments were also exposed to AR1/MB to determine the presence of the functional groups post-dyeing.

Results

Macroscopically, a yellowish hue was evident after Cipro-dyeing into the bD-PU segments. bD-PU segments dipped into Cipro, but not heated, had no gross color change. Exposure of pre- and post-dyed bD-PU segments to MB and AR1 had no visible difference in dye uptake (data not shown), demonstrating that the functional groups which would be utilized for protein binding were not affected by the lower temperature dyeing. The next step was to assess the antimicrobial activity of the bD-PU-AB material.

### Stage C: Demonstrating the Antimicrobial Activity of bD-PU-AB Segments (Acute Study)

Methods

Untreated, Cipro-dipped and dyed bD-PU segments were then cut into 1 cm² pieces, weighed and grouped into 3 segments/time interval. Segments were then washed in PBS on a rotary mixer at 37° C. for time intervals of 0, 1, 4, 24, 48, 72 and 120 hours. The wash solutions were changed at each time interval. The 1 cm² area bD-PU-AB segments were removed at each respective time interval and examined for antimicrobial activity using a zone of inhibition assay.

A stock solution of S. epidermidis was thawed at 37° C. for 1 hour. Upon thawing, 1 μl of this stock was added to 10 ml of Trypticase Soy Broth (TSB) and incubated overnight at 37° C. From this solution, 10 μl was streaked onto Trypticase Soy Agar (TSA) plates. Control and bD-PU-AB segments were then embedded into the streaked TSA plates (n=3 segments/time interval/treatment) and placed into a 37° C. incubator overnight. Standard 5 μg Cipro Sensi-Discs (n=3) were also embedded at each time interval as a positive control. The zone of inhibition each piece was determined, taking the average of 3 individual diameter measurements. Zone inhibition size (mm) over time was determined for each parameter evaluated.

Results

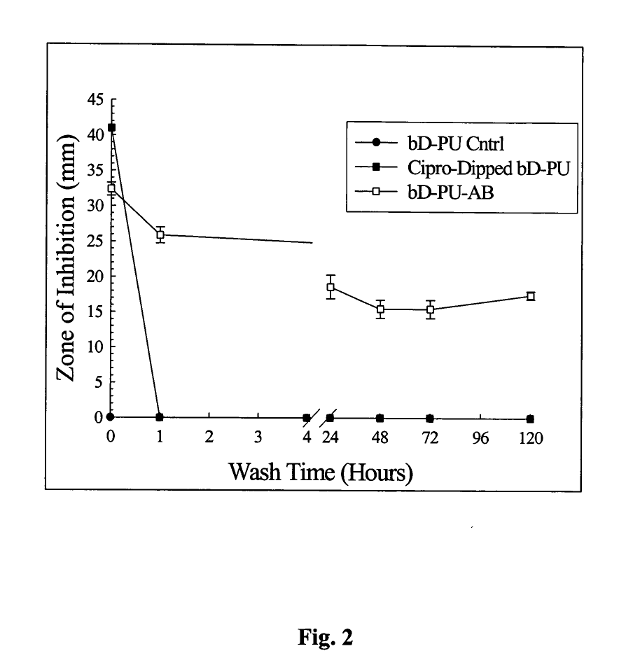

bD-PU-AB segments showed significant antimicrobial activity over the 5 days (length of study) as compared to the untreated and dipped controls, respectively. This graphically shown by FIG. 2. Cipro-dipped bD-PU material had an initial zone of inhibition prior to washing. However, this initial antimicrobial activity was lost within 1 hour of washing. Untreated materials, which are the current standard of clinical care, had no antimicrobial activity when evaluated at any of the examined time periods.

Although solution-dyeing technology was not successful for applying Cipro to unmodified Dacron fiber, this solution-dyeing technique—in conjunction with autoclaving—was effective and efficient in incorporating Cipro into the bifunctionalized material, with an observed subsequent release of the surface bound antibiotic which extended over 40 days in duration. This slow release of initially bound Cipro is comparable to that observed previously with the pad-heating process upon unmodified Dacron. This dyeing/autoclaving technique also was found to preserve the functional groups then existing on the fibrous material surface due to a reduced exposure to high heat; and this technique also permits and provides for subsequent chemical modification of then immobilized antibiotic with one or more covalently binding proteins.

This study clearly demonstrates that solution dyeing of Cipro into prepared bD-PU segments results in a surface that possesses significant antimicrobial activity as well as a surface that maintains the (EDA) bifunctional groups existing within the fibrous polymer. The next step was to examine the effects of both the EDA treatment and subsequent dyeing on the physical properties of the D-PU material.

### Stage D: Physical Characterization of bD-PU and bD-PU-AB Fibrous Matter Surfaces

Tensile Strength/Ultimate Elongation

Rectangular segments (5 cm×3 cm) of scoured knitted D-PU fibrous matter, bD-PU material and bD-PU-AB composition were cut from both the warp and weft directions from the larger sheets (n=4 segments/test group, total=8 segments). All cut segments were then evaluated for tensile strength and for ultimate elongation, similar to the previously conducted Dacron studies.

A Q-Test Apparatus was calibrated according to manufacturer's specifications in a temperature-controlled environment (temperature=23 C, 75% humidity). Each segment was then placed into two clamps spaced 2.0 cm apart, with 1.5 cm of each segment inserted into the clamp. Material stretching was initiated with a cross head speed of 12 in/min and terminated upon observation of visible tearing. The force required to “break” Dacron materials as well as the ultimate elongation (% stretch at break) was determined.

Results

Tensile strength of the knitted EDA-treated D-PU segments was reduced 38% in the warp (longitudinal) direction and 50% in the weft (width) direction as compared to unmodified D-PU segments. This strength loss is comparable to that observed when woven Dacron was exposed to EDA (40% strength loss) for a similar time period (data not shown). There was no further strength loss upon Cipro-dyeing of the bD-PU material. Ultimate elongation was reduced by 33% in the warp direction and 25% in the weft direction as compared to unmodified D-PU. Again, this loss in elongation was comparable (38%) to the EDA-treated woven Dacron segments (data not shown). Similar to the tensile strength results, no further loss in elongation further was obtained after Cipro-dyeing.

Exposure of bD-PU material to lower EDA concentrations and shorter reaction times did not significantly alter this initial loss in tensile strength/ultimate elongation. Elongation was significantly greater in the weft direction (2 and 2.2 fold, respectively) as compared to the warp direction for both control and EDA modified materials. This elongation is the direct result of inlaying polyurethane within the knitted structure in one direction. This stretch capacity would provide adequate elasticity for hernia repair mesh in a wound dressing or for an implantable dressing article/device in which a unidirectional compliance is necessary. The next step was to determine if a biologically-active protein could be covalently attached independently to the bD-PU-AB surface.

### Stage E: Independent Covalent Linkage of Thrombin onto Cipro-Dyed bD-PU Segments

Methods

bD-PU-AB segments (segment size=1 cm², n=8 segments/test group, 2 groups) were prepared as stated above herein. A stock 50 mM sodium bicarbonate buffer solution (pH 8.5) was utilized. A 20 mg/ml solution of Traut's reagent was prepared in the bicarbonate buffer and 2 ml was added to one set (n=4 segments/set, 2 sets) of bD-PU-AB segments in 20 ml borosilicate glass vials. To the other set, 2 ml of bicarbonate buffer was added to each set of segments (2 sets). All segment sets were then reacted for 1 hour on the orbital shaker at 120 rpm at room temperature.

Within 20 minutes of completion, a 45 μM 125I-thrombin solution (10% ¹²⁵I-thrombin) was prepared. Sulfo-SMCC (1 mg/ml; 71 μl) was added to the 125I-thrombin solution and reacted for 20 minutes at 37° C. in a water bath. The ¹²⁵I-thrombin-SMCC intermediate was then purified via gel filtration (PD-10 fast desalting column) and peak fractions pooled. The pooled ¹²⁵I-thrombin-SMCC solution was diluted to a final concentration of 13 μM.

Once the Traut's reaction was complete, all segments under test were washed twice on the inversion mixer with 5 ml bicarbonate buffer. The ¹²⁵I-thrombin-SMCC solution (2 ml) was then added to each tube and allowed to react for 3 hours at room temperature on an orbital shaker (150 r.p.m.).

After reactive incubation, the test segments were removed and washed four times in 2 ml 0.01M sodium phosphate, 0.5M NaCl, 0.05% Tween 20, pH 7.4 buffer for 5 minutes/wash on inversion mixer. Wash buffer was changed between washes. Segments were then gamma counted. Using protein concentration determined via Lowry assay and gamma counts of a set 125I-thrombin volume (i.e. specific activity), the amount of ¹²⁵I-thrombin (ng)/bD-PU-AB segment (mg) was determined.

Results

Incubation of the bD-PU-AB segments with Traut's reagent resulted in 37% greater 125I-thrombin binding (762±102 ng ¹²⁵I-thrombin/mg bD-PU-AB, p=0.00005) as compared to bD-PU-AB segments incubated with only bicarbonate buffer. Sonication is typically utilized to remove non-specific protein binding. However, a combination of the wash buffer and sonication was shown to elute Cipro from the material (data not shown). Accordingly, only detergent washing was employed, which may have resulted in higher non-specific binding for the control segments.

Incorporating the weight of the Dacron segments when determining total ¹²⁵I-thrombin binding permitted a normalization of the ¹²⁵I-thrombin binding data across deviations in the size of the segments. The next steps were to determine if the bD-PU-AB surface maintained antimicrobial activity and if the surface bound ¹²⁵I-thrombin was biologically-active.

### Experimental Series 2: Determination of Antimicrobial Activity for bD-PU-AB with Covalently Bound Thrombin

Methods

Untreated D-PU matter, unwashed bD-PU-AB material, bD-PU-AB segments that underwent all solution processes without protein exposure, bD-PU-AB segments with non-specifically bound ¹²⁵I-thrombin, and bD-PU-AB segments with covalently bound ¹²⁵I-thrombin were all individually evaluated for antimicrobial activity (n=4 segments/test condition) using the test procedures described above.

Results

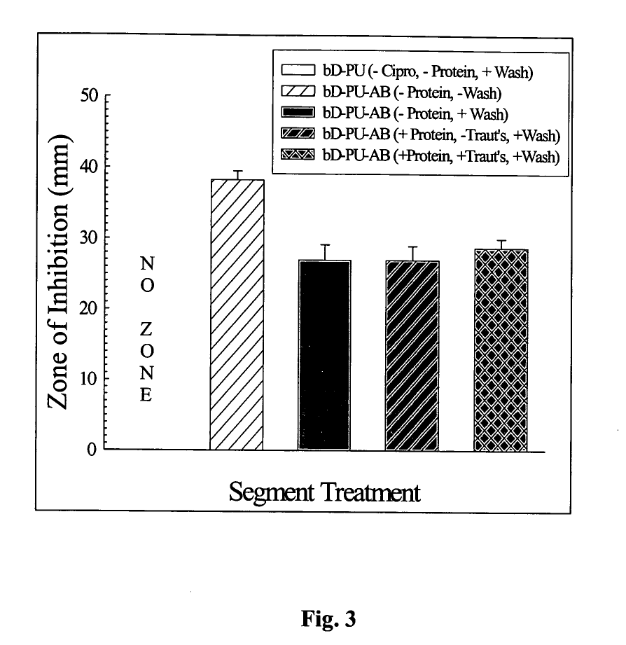

bD-PU-AB segments with covalently immobilized ¹²⁵I-thrombin had comparable antimicrobial activity (29±1.3 mm) to bD-PU-AB segments that went through all of the solutions of the linkage procedure but were not exposed to protein (27±2 mm; p=0.21), and to bD-PU-AB segments that had non-specifically bound 125I-thrombin (27±2 mm; p=0.19). This is illustrated by FIG. 3.

Untreated bD-PU segments, similar to the results obtained earlier herein, had no antimicrobial activity. The antimicrobial activity from the unwashed Cipro-dyed bD-PU-AB (38±1.3 mm) was greater than these three bD-PU-AB segment treatments, which were exposed to various solutions throughout the experimental procedure.

Thus, this empirical data clearly demonstrates that although Cipro concentration is decreased in each type of prepared material (via elution due to the various solution incubations), protein attachment does not itself cause or further decrease the antimicrobial activity of the differently prepared fibrous surfaces. Additionally, the level of antimicrobial activity generated from the bD-PU-AB segments with covalently bound ¹²⁵I-thrombin is still significant. The next step was to determine surface thrombin activity.

### Experimental Series 3: Examination of Surface Thrombin Activity Using a Chromogenic Assay

Methodology

Thrombin activity by untreated bD-PU-AB segments, by bD-PU-AB segments with non-specifically bound ¹²⁵I-thrombin, and by bD-PU-AB segments with covalently bound ¹²⁵I-thrombin was then determined (n=4 segments/treatment) using a chromogenic assay. The different segments were added into the bottom of a 1 cm path length UV/VIS cuvette (1 segment/cuvette). Tris buffer (0.01M Tris, 0.1M NaCl, 0.1% BSA, pH 7.4) was then added to bring the total volume to 1 ml. For thrombin standard solutions, a stock thrombin solution (0.6 ng/ml) was prepared in Tris buffer.

From this stock solution, 1 and 2.0 NIHU of ¹²⁵I-thrombin was added to each respective cuvette and brought up to a total volume of 1 ml. All cuvettes were incubated for 5 minutes in a Beckman spectrophotometer containing a thermocirculator, which regulated the cuvette chamber temperature to 37° C., and a six-chamber cuvette holder. After incubation, thrombin activity was then measured upon addition of 1 ml of 100 μM S-2238 by monitoring the change in absorbance per minute at 15-second intervals for 3 minutes at 410 nm.

Results:

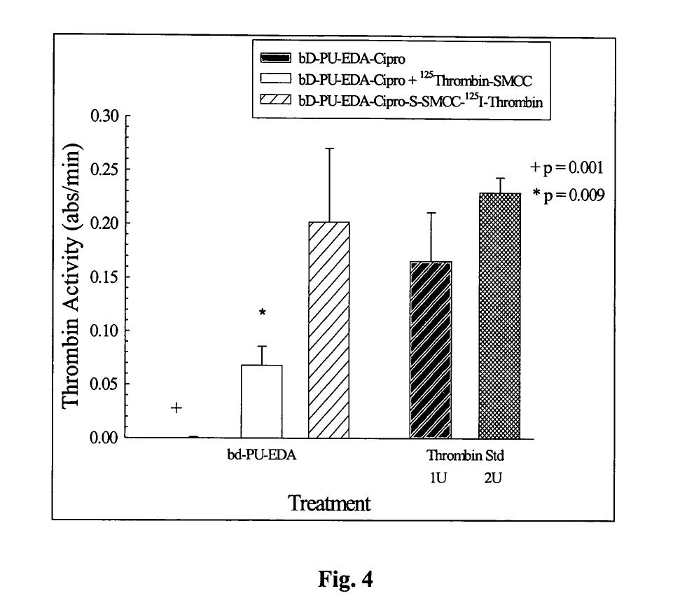

bD-PU-AB segments with covalently bound ¹²⁵I-thrombin had 3-and 505-fold greater surface thrombin activity [0.202±0.07 absorbance (Abs)/minute] as compared to bD-PU-AB with non-specifically bound ¹²⁵I-thrombin (0.068±0.02 Abs/minute) and untreated bD-PU (0.0004±0.0006 Abs/minute). This is illustrated by FIG. 4.

All fibrous matter medical devices will adsorb proteins from the host, with blood-contacting fibrous articles and implants having a far greater exposure to and absorbance of the various proteins in the body fluids. This protein adsorption phenomenon and characteristic has been taken into account when designing this in vitro experiment.

Accordingly, thrombin activity was evaluated in the presence of 0.1% albumin in an effort to mimic thrombin activity upon exposure in vivo. The empirical results revealed that protein adsorption by the fibrous dressing did not adversely affect the enzymatic activity of thrombin under these conditions. Overall, the tested bD-PU-AB segments with covalently bound thrombin had greater enzymatic activity than the control segments, even when non-specifically bound protein is present, and while showing effective and comparable antimicrobial activity.

### Experimental Series 4: Assessment of Surface Thrombus Formation by Various bD-PU Segments Upon Exposure to Whole Blood

Methodology

Surface thrombus formation on control segments (anionic Dacron, Hyd), and EDA-treated Dacron dyed with Cipro (EDA-Cipro), and test (EDA-treated Dacron dyed with Cipro followed by surface thrombin immobilization (EDA-Cipro-Thrombin) segments were evaluated after exposure to whole blood.

Whole blood (18 ml) was collected by a phlebotomist at the time of the experiment from a healthy male volunteer using a 22G butterfly needle. Blood was drawn into a syringe containing 2 ml of 3.8% sodium citrate, resulting in a final sodium citrate concentration of 0.38% (v:v). This citrated whole blood (1 ml volume) was then transferred into borosilicate tubes and temperature maintained at 37° C. via a heating chamber. Control and test segments, which were placed onto metal hooks to permit rapid removal from the assay, were then inserted into individual tubes containing citrated whole blood.

All segments were then removed after an exposure time of 1 minute. Exposure times of 2.5 and 5 minutes were also assessed using unexposed control and test segments, as well as 1 ml of fresh citrated whole blood. One set of blood tests (n=2) was also conducted which contained only citrated blood, followed by the addition of native thrombin (1 NIHU), a concentration employed in the surface immobilization studies (the positive control).

All segments were immediately removed after the reactive incubation time, rinsed in PBS buffer (at 37° C.), and then observed for gross thrombus deposition. The segments were then fixed in 10% buffered formalin.

Afterwards, the segments were then randomly divided—with one portion being processed by routine histological analysis and the other portion processed by scanning electron microscopy (SEM) using a JEOL JSM 5900LV electron microscope.

Results

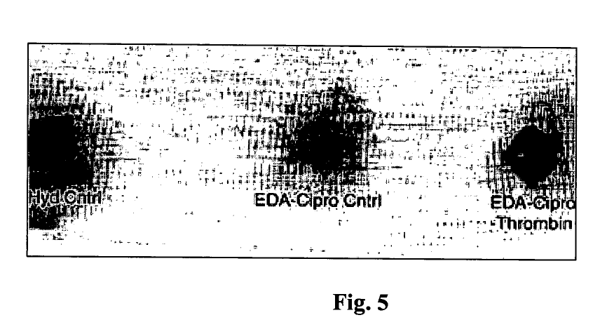

Surface thrombus deposition was macroscopically apparent on the EDA-Cipro-Thrombin segments after 2.5 minutes of exposure to whole blood, with significant thrombus formation present after 5 minutes time. This is clearly shown by FIG. 5.

In contrast, both the Hyd and EDA-Cipro controls had no apparent gross thrombus formation over any of the time periods examined. Only red cell attachment was apparent on the negative control materials. In contrast, the positive controls did show rapid thrombus formation. These results were qualitatively confirmed via histological analysis as well as by the SEM analysis.

Histological assessment of the EDA-Cipro-Thrombin segments revealed significant thrombus formation, which was comprised of red cells, platelets and fibrin, within the fibers and on the surface. In contrast, the EDA-Cipro and Hyd controls had no apparent thrombus formation throughout any of the time periods evaluated.

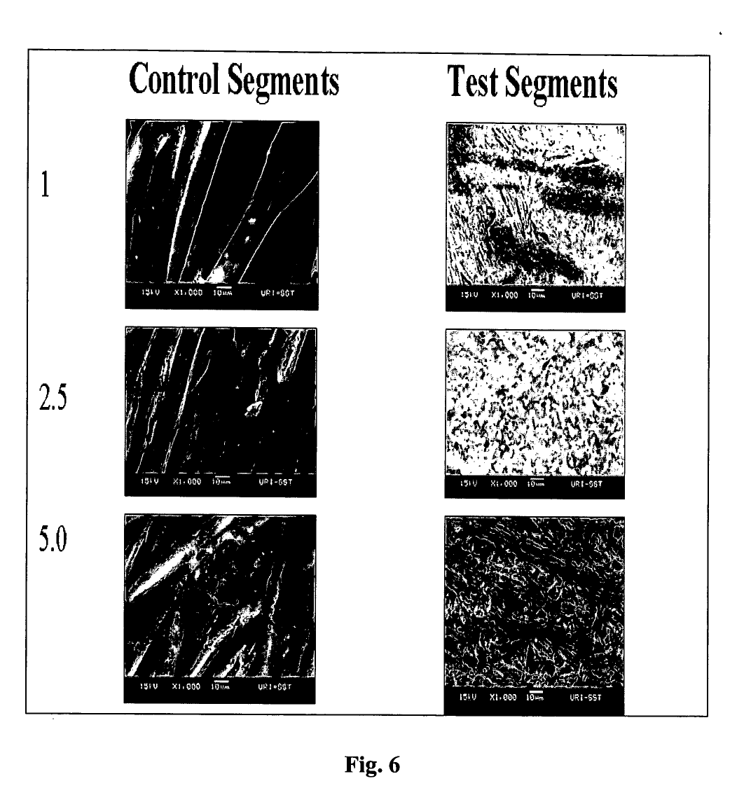

Scanning electron microscopy of the EDA-Cipro-Thrombin surfaces showed the time sequence of thrombus formation. This is illustrated by FIG. 6. Although no thrombus was grossly apparent on the EDA-Cipro-Thrombin surfaces at 1 minute's exposure time, the SEM analysis revealed the initiation of fibrin formation within the Dacron matrix at this same time period. Thrombus deposition then became more pronounced and organized after 2.5 and 5 minutes, respectively. However, no thrombus was evident at any of the time periods for the EDA-Cipro or Hyd control segments. Only blood protein adhesion with scattered red cell deposition was observed for these control segments.

The present invention is not to be restricted in form nor limited in scope except by the claims appended hereto.

2. A fabric article useful as a wound dressing, said fabric article comprising:
preformed fibrous matter of predetermined dimensions and configuration, said fibrous matter having at least one exterior surface and being formed of at least one type of material able to take up aqueous fluids;
a coupling agent attached to at least the exterior surface portion of said preformed fibrous matter;
at least one water-miscible antibiotic composition of fixed concentration which is incorporated into said fibrous matter in the dry state, but which becomes detached from said fibrous matter and is released into the ambient environment as a freely mobile antibiotic composition after said fibrous matter takes up an aqueous fluid, said antibiotic composition having recognized characteristic antimicrobial properties in the immobilized and freely mobile forms, being a heat stable substance, and having a relative molecular mass in the 300-1500 range;
at least one bifunctional cross-linking molecule joined to said coupling agent at said exterior surface of said fibrous matter; and
a prechosen biologically active protein covalently bound by said bifunctional cross-linking molecule to said fibrous matter, said protein having recognized biologically active properties for aiding the wound healing process while bound to said fibrous matter.

3. The fabric article as recited in claim 1 or 2 wherein said fibrous matter is a woven material.

4. The fabric article as recited in claim 1 or 2 wherein said fibrous matter is a non-woven material.

5. The fabric article as recited in claim 1 or 2 wherein said fibrous matter comprises at least one naturally occurring fibrous material.

6. The fabric article as recited in claim 1 or 2 wherein said fibrous matter comprises at least one synthetic fibrous material.

7. The fabric article as recited in claim 2 wherein said coupling agent is ethylenediamine.

8. The fabric article as recited in claim 2 wherein said coupling agent is selected from the group consisting of polyethylenimines, (polypropyleneglycol) bis(2-Aminopropyl ether), 1,3 propylenediamine, 1,2 propylenediamine, neopentadiamine, butylenediamine, pentylenediamine, hexamethylenediamine, octamethylenediamine, diethylenetriamine, N-(2-aminopropyl)-1,3-propanediamine,-(3-aminopropyl)-1,3-propanediamine, N,N-1,2-ethylene bis(1,3-propanediamine), and tetraethylenepentamine.

9. The fabric article as recited in claim 1 or 2 wherein said antibiotic composition has recognized broad spectrum antimicrobial properties.

10. The fabric article as recited in claim 1 or 2 wherein said antibiotic composition is a fluoroquinolone.

11. The fabric article as recited in claim 1 or 2 wherein said antibiotic composition is ciprofloxacin.

12. The fabric article as recited in claim 1 or 2 wherein said antibiotic composition is selected from the group consisting of Ofloxacin, Norfloxacin, Sparfloxacin, Tomafloxacin, Enofloxacin, Lovafloxacin, Lomefloxacin, Pefloxacin, Fleroxacin, Avefloxin, and DU6859a.

13. The fabric article as recited in claim 1 or 2 wherein said protein is a blood coagulation cascade protein.

14. The fabric article as recited in claim 1 or 2 wherein said protein is a cytokine selected from the group consisting of interleukins, interferons, tumor necrosis factor, and granulocyte colony stimulating factor.

15. The fabric article as recited in claim 1 or 2 wherein said protein is a growth factor.

16. The fabric article as recited in claim 1 or 2 wherein said protein is thrombin.

17. The fabric article as recited in claim 1 or 2 wherein said fabric article is fashioned as a wound dressing for topical and subtopical use.

18. The fabric article as recited in claim 1 or 2 wherein said fabric article is fashioned as a wound dressing suitable for percutaneous, transcutaneous or subcutaneous use.

19. The fabric article as recited in claim 1 or 2 wherein said fabric article is fashioned as a wound dressing for internal use within the body of a living subject.

20. The prepared fabric article as recited in claim 1 or 2 wherein said fabric article has been pre-packaged and pre-sterilized.

21. A method for making a fabric article useful as a wound dressing, said method comprising the steps of:
obtaining a preformed fibrous matter comprised of at least one chemical composition which provides multiple chemical groups functional for subsequent chemical reaction, said fibrous matter being of determinable dimensions and configuration, having at least one exterior surface which presents a plurality of functional chemical groups for chemical reaction, and being formed of at least one type of material able to take up aqueous fluids;
preparing a heated aqueous antibiotic fluid of predetermined concentration at a temperature greater than 20° C. and less than 100° C. comprising water and at least one water-miscible antibiotic composition which has characteristic antimicrobial properties, is heat stable and has a relative molecular mass in the 300-1500 range;
immersing said preformed fibrous matter into said heated aqueous antibiotic fluid of predetermined concentration for a predetermined time period not less than about 30 minutes duration;
autoclaving said antibiotic immersed preformed fibrous matter at a temperature above 130° C. for a time period not less than about 10 minutes in duration whereby
(i) said antibiotic composition becomes incorporated onto the exterior surface of said fibrous matter, and
(ii) said antibiotic composition becomes detached from and is released into the ambient environment as a freely mobile composition after said fibrous matter takes up an aqueous fluid,
(iii) said antibiotic composition retains its recognized characteristic antimicrobial properties in the immobilized and freely mobile states; and
introducing at least one prechosen biologically active protein having recognized properties for aiding the wound healing process to said exterior surface of said fibrous matter, whereby
(a) said introduced protein reacts with and becomes covalently bound to said fibrous matter, and
(b) said protein retains its recognized biologically active properties for aiding the wound healing process while being covalently bound to said fibrous matter.

22. A method for making a fabric article useful as a wound dressing, said method comprising the steps of:
obtaining a preformed fibrous matter of predetermined dimensions and configuration, having at least one exterior surface, and being formed of at least one type of material able to take up aqueous fluids;
applying a coupling agent to said fibrous matter whereby said coupling agent reacts with and becomes attached to at least the exterior surface portion of said fibrous matter;
preparing a heated aqueous antibiotic fluid of predetermined concentration at a temperature greater than 20° C. and less than 300° C. comprising water and at least one water-miscible antibiotic composition which has characteristic antimicrobial properties, is heat stable and has a relative molecular mass in the 300-1500 range;
immersing said preformed fibrous matter and attached coupling agent into said heated aqueous antibiotic fluid of predetermined concentration for a time period not less than about 30 minutes in duration;
autoclaving said antibiotic immersed preformed fibrous matter and joined bifunctional binding agent at a temperature above about 130° C. for a time period not less than about 10 minutes in duration whereby
(i) said antibiotic composition becomes incorporated onto the exterior surface of said fibrous matter in the dry state, and
(ii) said antibiotic composition becomes detached from said fibrous matter agent and is released into the ambient environment as a freely mobile composition after said fibrous matter takes up an aqueous fluid,
(iii) said antibiotic composition retains its recognized characteristic antimicrobial properties in the immobilized and freely mobile forms;
joining at least one bifunctional cross-linking molecule to said external surface of said fibrous matter; and
introducing at least one prechosen biologically active protein having recognized properties for aiding the wound healing process to said exterior surface of said fibrous matter, whereby
(a) said introduced protein reacts with said joined bifunctional cross-linking molecule and becomes covalently bound to said fibrous matter, and
(b) said prechosen protein retains its recognized biologically active properties for aiding the wound healing process while covalently bound to said fibrous matter.

23. The method for making a fabric article as recited in claim 21 or 22 wherein said antibiotic composition comprises at least one ring structure.

24. The method for making a fabric article as recited in claim 21 or 22 wherein said antibiotic is a fluoroquinolone.

25. The method for making a fabric article as recited in claim 21 or 22 wherein said antibiotic composition is selected from the group consisting of anti-bacterial and anti-fungal agents.

26. The method for making a fabric article as recited in claim 21 or 22 wherein said antibiotic composition is selected from the group consisting of Ciprofloxacin, Ofloxacin, Norfloxacin, Sparfloxacin, Tomafloxacin, Enofloxacin, Lovafloxacin, Lomefloxacin, Pefloxacin, Fleroxacin, Avefloxin, and DU6859a.

27. The method for making a fabric article as recited in claim 21 or 22 wherein said fibrous matter comprises a synthetic polymer material.

28. The method for making a fabric article as recited in claim 21 or 22 wherein said fibrous matter comprises a naturally-occurring material.

29. The method for making a fabric article as recited in claim 21 or 22 wherein said fibrous matter comprises non-woven material.

30. The method for making a fabric article as recited in claim 21 or 22 wherein said fibrous matter comprises woven material.

31. The method for making a fabric article as recited in claim 21 or 22 wherein said biologically active protein is a blood coagulation cascade protein.

32. The method for making a fabric article as recited in claim 21 or 22 wherein said biologically active protein is a cytokine.

33. The method for making a fabric article as recited in claim 21 or 22 wherein said biologically active protein is a growth factor.

34. The method for making a fabric article as recited in claim 21 or 22 wherein said biologically active protein is thrombin.
